(19) United States
(12) Patent Application Publication
(10) Pub. No.: US 2022/0025316 A1
(43) Pub. Date: Jan. 27, 2022

(54) TRITURATION DEVICES FOR TISSUE DISAGGREGATION

(71) Applicant: Prim Sigma Technologies, Inc., Beverly Hills, CA (US)

(72) Inventors: Arthur Yajing YU, Arcadia, CA (US); Jian Jim YE, Monterey Park, CA (US)

(21) Appl. No.: 16/935,639

(22) Filed: Jul. 22, 2020

(51) Int. Cl. C12M 1/33; G01N 1/28

(52) U.S. Cl. CPC C12M 45/02; G01N 1/286

(57) ABSTRACT

A trituration device for tissue disaggregation includes a device housing having a hollow cylinder defining an opening extending from a first end of the device housing to a second end of the device housing, a rotor block positioned in the opening of the device housing, and a trituration grater positioned within the opening of the device housing to contact the rotor block. The rotor block includes a solid cylinder having a central channel and at least one surface that defines a rotor blade, and the trituration grater includes multiple angled cutting holes. The device also includes a collection vessel positioned at the second end of the device housing to collect disaggregated tissue of a tissue specimen, and an actuating stem extending through the central channel of the rotor block to rotate the rotor block in response to rotation of the actuating stem.

### FIELD

The present disclosure generally relates to trituration devices for tissue disaggregation, and methods of using a trituration device for tissue disaggregation.

### BACKGROUND

This section provides background information related to the present disclosure which is not necessarily prior art.

In the field of laboratory research and clinical medicine, plant or animal tissue disaggregation may be required prior to proceeding with further steps in the research or applications. Many existing tissue processing devices are either for single cell preparations, or are inadequate to treat tissues. Additionally, some tissue samples have limited availability, and may be too tough to be handled by these tissue processors. Unfortunately, many tissue samples such as skin, tendons, scalp tissue, nerve fibers, periosteum tissue, blood vessels, plant stems and roots, etc., belong to this “tough tissue” category, and the yield of biopsy or sampling of these specimens may be so small that often only a few hundred micrograms or less are available.

Further, not all tissues need to be ground down to single cells. In some cases, procuring cell aggregates of a few dozen cells or more in their original forms and shapes might be advantageous for the purpose of mimicking their original organelle functionality, because the specific microenvironment or niche of the original organ or tissue is maintained. Consequently, they could potentially be used clinically as micrografts to promote tissue regeneration in situ. One area that might have great use of such “micrografts” would be alopecia, which affects millions of men and women. Other areas of potential application could be myocardial infarction treatment using cardiac appendage tissue, wound healing using Wharton's Jelly, bone grafting enhancement using periosteum micrografts, etc. However, existing tissue processing devices are inadequate based on limited amounts of samples, tough tissues, and inability to process tissues into cell aggregates for use as “micrografts,” etc.

Tissue disaggregation is usually the first step for successful tissue cultures. Typically, the tissue is treated mechanically or enzymatically, resulting in free single cell suspensions. However, in many cases enzymatic tissue disaggregation may not be preferred due to subsequent changes in cell characteristics, among other factors. Example enzymatic tissue disaggregation is described by Joel A. Aronowitz J A, Lockhart R A, and Hakakian C S. Mechanical versus enzymatic isolation of stromal vascular fraction cells from adipose tissue. Springerplus. 2015; 4:713. See also Senesi L, De Francesco F, Farinelli L, Manzotti S, Gagliardi G, Papalia G F, Riccio M and Gigante A. Mechanical and Enzymatic Procedures to Isolate the Stromal Vascular Fraction From Adipose Tissue: Preliminary Results. Front Cell Dev Biol. 7 Jun. 2019; 7:88.

On the other hand, one of the main advantages of mechanically disaggregating tissues is that the cells obtained may be able to maintain their original characteristics, thus facilitating further studies and helping to achieve better therapeutic goals. Many suitable methods may be used for mechanical tissue disaggregation, including cutting, scissoring, sieving, a mortar and pestle, a bead mill, trituration, a homogenizer, a food processor, a screw press, sonication, etc. Example methods for tissue disaggregation are described in U.S. Pat. No. 2,955,530; U.S. Pub. 2018/0236457; U.S. Pat. No. 5,731,199, Int. Pub. WO2015005871; Int. Pub. WO2014000031; U.S. Pat. No. 8,440,440; and Quatromoni J G, Singhal, S, Bhojnagarwala P, Hancock W W, Albelda S M, and Eruslanov E. An optimized disaggregation method for human lung tumors that preserves the phenotype and function of the immune cells. J Leukoc Biol. 2015 January; 97(1): 201-209. Online 30 Oct. 2014.

Some of the tissues consist of cells that are tightly aggregated. Tissues such as epithelium, especially the scalp, have strong binding between cells and connective tissues. Further, some tissues contain a large proportion of tightly woven collagen fibers. These factors make mechanical tissue disaggregation extremely difficult. In this situation, the tissue is sliced with a blade or scissors into smaller pieces first, then the pieces are further processed with other mechanical methods to achieve finer tissue disaggregation, usually until single cell suspension is obtained. With an appropriately chosen method, mechanical tissue disaggregation may produce a good cell yield and a good cell survival, without being inferior to the enzymatic approaches.

Another important consideration is the current official regulations. Regenerative medicine has been receiving more and more attention in the last twenty years. As a result, obtaining stem cells from all kinds of tissues is an essential step for many relevant studies and applications. However, FDA regulations mandate that for a cellular treatment to be considered safe and acceptable, the cells must be treated with “minimal manipulation” and be subjected to “same use,” similar to the situation where a skin graft is used clinically: the skin graft is harvested from a donor site on the same person with a mechanical instrument (a blade or a skin drum), followed by implanting it to a denuded area. During the process, the skin graft is minimally manipulated, and then the graft is used for the same purpose: covering an area of body surface as it is meant to.

While mechanical treatment of a tissue is generally considered as “minimal manipulation,” enzymatic or chemical treatment are deemed “extensive manipulation.” Consequently, for a cell therapy to obtain an approval from the FDA, a mechanical approach should be employed to disaggregate the tissue samples. Otherwise, samples from enzymatic or chemical treatment could only be used for laboratory research, unless an investigational new drug (IND) protocol is obtained.

Existing mechanical means for tissue disaggregation include a screw press, a bead mill, a food processor, a triturator, a mortar and pestle, etc., but many known tissue disaggregation devices have limited use for various reasons as described above. Some approaches and technologies have been developed recently in the field of regenerative medicine, such as hair regeneration, as described by Talavera-Adame D, Newman D, and Newman N. Conventional and novel stem cell based therapies for androgenic alopecia. Stem Cells Cloning. 2017; 10:11-19. Online 31 Aug. 2017. See also Castro A R, Logarinho E. Tissue engineering strategies for human hair follicle regeneration: How far from a hairy goal? Stem cells translational medicine, Volume 9(3) March 2020: 342-350. And see Owczarczyk-Saczonek A, Krajewska-Wlodarczyk M, Kruszewska A, Lukasz Banasiak, Placek W, Maksymowicz W, and Wojtkiewicz W. Therapeutic Potential of Stem Cells in Follicle. Regen Stem Cells Int. 2018; 1049641. Online 5 Aug. 2018.

Some approaches have utilized hair stem cells cultured from laboratories on animal models, with mixed success. While attempts to grow hair with single cell suspensions have failed repeatedly, works by Toyoshima et al. demonstrated that bioengineered hair follicular microstructures can successfully grow normal hair, after being transplanted into mice. See Toyoshima K E, Asakawa K, Ishibashi N, Toki H, Ogawa M, Hasegawa T, Irié T, Tachikawa T, Sato A, Takeda A & Tsuji T. Fully functional hair follicle regeneration through the rearrangement of stem cells and their niches. Nature Communications 3:784:1-13. The bioengineered follicle organelles also restored hair growth cycles and piloerection through the rearrangement of follicular stem cell microstructures and their niches. This and the work by others on transplantation of hair follicle organelles suggest that original hair follicle cell aggregates or niches may work better than single cell suspensions (primary or cultured) for hair regeneration, as described by AlSogair S S. Stem cell therapy and hair loss: Present evidence and future perspectives. J. Derm. Derm Surg. (2019) 23(2):61-65. See also Owczarczyk-Saczonek A, Krajewska-Wlodarczyk M, Kruszewska A, Lukasz Banasiak, Placek W, Maksymowicz W, and Wojtkiewicz W. Therapeutic Potential of Stem Cells in Follicle. Regen Stem Cells Int. 2018; 1049641. Online 5 Aug. 2018. The mechanism may be due to the fact that hair growth is a carefully orchestrated effort of the assortment of cells in the hair follicle bulb structures. Single cells, no matter how potent, simply may not start or promote the hair growth in areas where hair follicles have entered a temporary or permanent resting state, as seen in various alopecia conditions.

Some tissues that have been studied with tissue disaggregation include the periosteum, the cardiac atrial appendage, and the lateral rectus muscle of the eyes, as described by Trovato L, Monti M, Del Fante C, Cervio M, Lampinen M, Ambrosio L, Redi C A, Perotti C, Kankuri E, Ambrosio G, Rodriguez Y, Baena R, Pirozzi G, and Graziano A. A New Medical Device Rigeneracons Allows to Obtain Viable Micro-Grafts from Mechanical Disaggregation of Human Tissues. J Cell Physiol. October 2015; 230(10):2299-303. This study shows that the coarsely disaggregated tissues have good cell viability and are suitable for tissue transplant as micrografts. Additionally, the disaggregated tissues/cells maintained the expected cellular markers and all other characteristics. This finding is consistent with the unique properties and clinical importance of the cell aggregates/niches, for their ability to mimic the original functional state in situ.

Along the same line, a clinical application of the micrografts using hair follicular tissue was reported with promising results, by Gentile P, Scioli M G, Bielli A, Orlandi A, and Cervelli V. Stem cells from human hair follicles: first mechanical isolation for immediate autologous clinical use in androgenetic alopecia and hair loss. Stem Cell Investig. 2017; 4:58. Published online 27 Jun. 2017. See also Gentile P. Autologous Cellular Method Using Micrografts of Human Adipose Tissue Derived Follicle Stem Cells in Androgenic Alopecia. Int J Mol Sci. 2019; 20(14):3446. Published 13 Jul. 2019. doi:10.3390/ijms20143446. A very thin piece of hair bearing scalp tissue was punch-biopsied and processed. The processed tissue becomes fragmented into aggregates of hair follicle stem cells along with other cells, such as fibroblasts and epithelial cells. The sizes of these micrografts/cell aggregates range from 80 to 140 micrometers (whereas single cells usually size from 10 to 40 microns). After the “micrografts” or cell aggregates were grafted back by injection into the scalp where hair was lost, the hair was found to be 27% thicker at 23 weeks post procedure. One of the major drawbacks of this study was that the cell productivity from this apparatus was extremely low: only 3728+/−664.5 cells (of all kinds) for the biopsied piece which weighs about 100 micrograms (calculated using the measurements provided by the author). Given the fact that, theoretically, a 50 to 100 microgram of tissue contains at least 5×10⁶ cells, it is somewhat disappointing that fewer than 0.076% of the cells were harvested from the punch-biopsy sample after close examination of the actual apparatus and the documents. See U.S. Pat. Pub. 2018/0236457, and Gentile P, Scioli M G, Bielli A, Orlandi A, and Cervelli V. Stem cells from human hair follicles: first mechanical isolation for immediate autologous clinical use in androgenetic alopecia and hair loss. Stem Cell Investig. 2017; 4:58. Published online 27 Jun. 2017. The main reasons for such an extremely low yield may include: (1) The thin, floating bladed rotor wobbles during the action and is not able to stably press down on the sample pieces for the trituration action; and (2) The fact that the triturating micro-holes (micro cutting blades) of the “disaggregating grid” are too few, and their pointy and small (80 micron) openings protrude straight upward for a long distance, essentially preventing an efficient trituration. When using the apparatus to prepare hair follicle micrografts, sample pieces prepared according to the original authors' protocol basically remained intact on the grid, except for a few scratches on the tissue surfaces. The number of harvested cells was also very low, at about the same level as presented by the authors. These same issues existed already in an older invention described in U.S. Pat. No. 5,731,199 issued to Gianmarco Roggero in 1994, and the design of U.S. Pub. 2018/0236457 filed by Antonio Graziano and Riccardo D'Aquino in 2015, was based on Rogerro's U.S. Pat. No. 5,731,199.

### SUMMARY

According to one aspect of the present disclosure, an example trituration device for tissue disaggregation includes a device housing having a hollow cylinder defining an opening extending from a first end of the device housing to a second end of the device housing, a rotor block positioned in the opening of the device housing, and a trituration grater positioned within the opening of the device housing to contact the rotor block. The rotor block includes a solid cylinder having a central channel and at least one surface that defines a rotor blade, and the trituration grater includes multiple angled cutting holes. The device also includes a collection vessel positioned at the second end of the device housing to collect disaggregated tissue of a tissue specimen, and an actuating stem extending through the central channel of the rotor block to rotate the rotor block in response to rotation of the actuating stem.

According to another aspect of the present disclosure, a trituration device for tissue disaggregation includes a device housing, a rotor block positioned in the device housing, and a trituration grater positioned in the device housing. The rotor block includes a central channel and at least one rotor blade, and the trituration grater includes multiple angled cutting holes. The device also includes a collection vessel positioned at an end of the device housing to collect disaggregated tissue of a tissue specimen, and an actuating stem including a first end, a second end opposite the first end, and a middle portion defined between the first end and the second end. A cross-sectional width of the middle portion is greater than a cross-sectional width of the first end and the second end, the actuating stem extends through the central channel of the rotor block, and a shape of the central channel of the rotor block corresponds to a shape of the cross-section of the middle portion of the actuating stem to facilitate rotation of the rotor block in response to rotation of the actuating stem. The collection vessel includes a central column defining an opening, and the second end of the actuating stem is received in the opening of the central column.

According to yet another aspect of the present disclosure, a method of disaggregating tissue using a trituration device is disclosed. The device includes a device housing, a rotor block comprising a solid cylinder including a central channel and at least one recess that defines a rotor blade, a trituration grater including multiple angled cutting holes, a collection vessel to collect disaggregated tissue, and an actuating stem. The method includes positioning a tissue specimen within the at least one recess of the rotor block, inserting the rotor block into the device housing to contact the trituration grater, while the tissue specimen remains in the at least one recess of the rotor block, inserting the actuating stem through the central channel of the rotor block, and rotating the actuating stem using a motor or crank to rotate the rotor block and press the tissue specimen against the trituration grater to receive disaggregated tissue in the collection vessel.

Further aspects and areas of applicability will become apparent from the description provided herein. It should be understood that various aspects of this disclosure may be implemented individually or in combination with one or more other aspects. It should also be understood that the description and specific examples herein are intended for purposes of illustration only and are not intended to limit the scope of the present disclosure.

### DRAWINGS

The patent or application file contains at least one drawing executed in color. Copies of this patent or patent application publication with color drawing(s) will be provided by the Office upon request and payment of the necessary fee.

The drawings described herein are for illustrative purposes only of selected embodiments and not all possible implementations, and are not intended to limit the scope of the present disclosure.

FIG. 1A is an exploded view of trituration device for tissue disaggregation, according to one example embodiment of the present disclosure.

FIG. 1B is an assembled view of the trituration device of FIG. 1A.

FIG. 2A is an exploded sectional view of the trituration device of FIG. 1A.

FIG. 2B is an assembled sectional view of the trituration device of FIG. 1A.

FIG. 3A is a top isometric view of the rotor block of the trituration device of FIG. 1A.

FIG. 3B is a bottom isometric view of the rotor block of the trituration device of FIG. 1A.

FIG. 4 is a top view of the trituration grater of the trituration device of FIG. 1A.

FIG. 5 is an exploded view of a portion of the trituration device of FIG. 1A, illustrating a location for loading a tissue specimen in the rotor block.

FIG. 6 is an exploded view illustrating an optional motor coupler for the trituration device of FIG. 1A.

FIG. 7 is an isometric view of a rotor block including a side wall in the tissue specimen loading area, according to another example embodiment of the present disclosure.

FIGS. 8 and 9 are top views of alternative trituration graters of the trituration device of FIG. 1A, illustrating different optional grater patterns.

FIG. 10 is an isometric view of a rotor block having a single tissue specimen loading area, according to another example embodiment of the present disclosure.

FIG. 11 is an isometric view of a rotor block having four tissue specimen loading areas, according to another example embodiment of the present disclosure.

FIG. 12 is an isometric view of the trituration device of FIG. 1, including bead mill in a collection vessel.

FIG. 13 is a magnified view of example hair follicle cell aggregates after processing by the trituration device of FIG. 1A.

FIGS. 14A and 14B are example photographs of a male patient before and after hair loss treatment using the trituration device of FIG. 1A.

FIG. 15 is a 40× magnified view of example cardiac atrial appendage cells after processing by the trituration device of FIG. 1A.

FIG. 16 is a 100× magnified view of example cardiac atrial appendage cells after processing by the trituration device of FIG. 1A.

FIG. 17 is a magnified view of periosteal cell aggregates after processing by the trituration device of FIG. 1A.

Corresponding reference numerals indicate corresponding (but not necessarily identical) parts throughout the several views of the drawings.

### DETAILED DESCRIPTION

Example embodiments will now be described more fully with reference to the accompanying drawings.

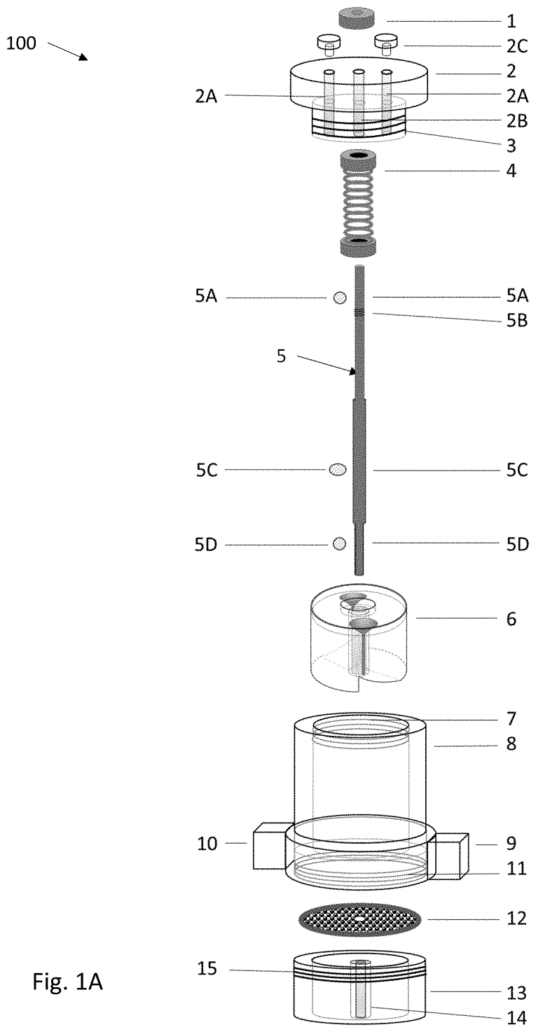

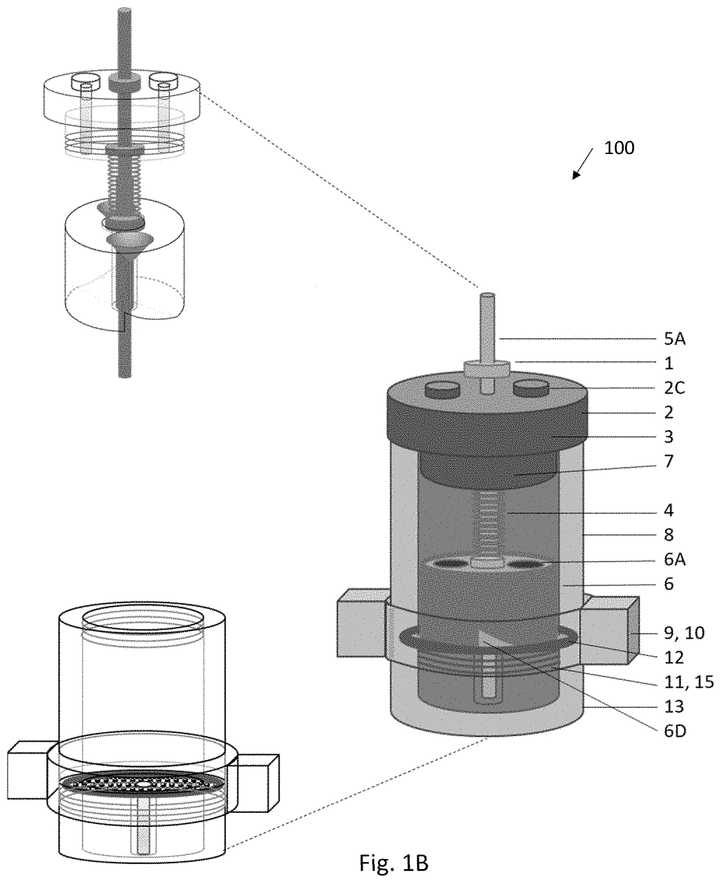

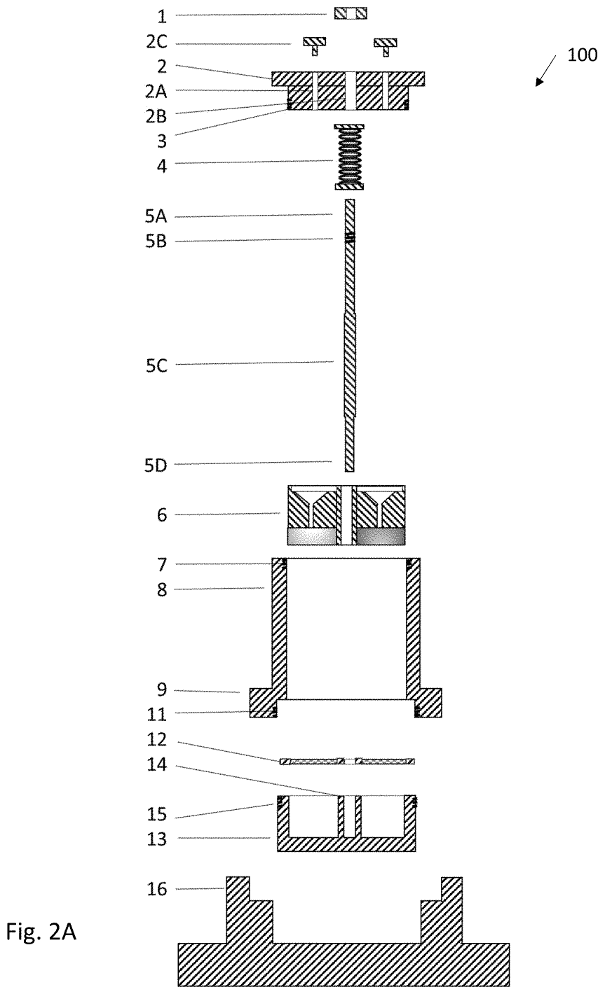

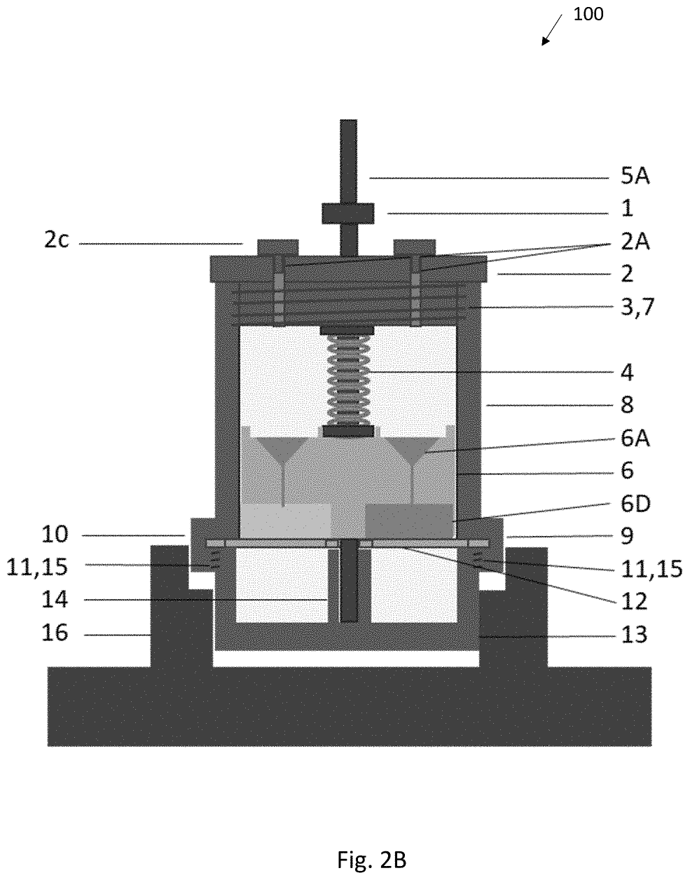

FIGS. 1A, 1B, 2A and 2B illustrate an example trituration device 100 for tissue disaggregation. The device 100 includes a device housing 8 having a hollow cylinder that defines an opening extending from a first end of the device housing 8 to a second end of the device housing 8. The device 100 also includes a rotor block 6 positioned in the opening of the device housing 8, and a trituration grater 12 positioned within the opening of the device housing 8 to contact the rotor block 6.

The rotor block 6 includes a solid cylinder having a central channel and at least one surface that defines a rotor blade (as described further below). The trituration grater 12 includes multiple angled cutting holes. The device 100 also includes a collection vessel 13 positioned at the second end of the device housing 8 to collect disaggregated tissue of a tissue specimen, and an actuating stem 5 extending through the central channel of the rotor block 6 to rotate the rotor block 6 in response to rotation of the actuating stem 5.

The device 100 may be generally considered as a micro triturator having an upper portion (e.g., half) including a solid cylindrical rotor 6, and a lower portion (e.g., half) housing the triturating grater 12 and the collection vessel 13 (e.g., compartment, etc.). FIG. 1A is an exploded transparent view of the device 100, while FIG. 1B is a transparent view illustrating the upper and lower portions of the device 100 being combined into the fully assembled device 100.

As shown in FIG. 1A, the actuating stem 5 extends through a center of the device 100. The actuating stem 5 has an upper round portion 5A that includes male screw threads for coupling with a female threaded nut 1. The actuating stem 5 also includes a middle oval-shaped portion 5C, and a lower round portion 5D that passes through the lower half of the device 100 and into a center of the collection vessel 13 to function as a stabilizing element of the device 100. FIG. 1A illustrates cross-sectional shapes of the portions 5A (round), 5C (oval-shaped), and 5D (round).

The actuating stem 5 passes through a spring 4, and a cap having an upper portion 2 and a lower portion 3. The nut 1 is twisted onto the male screw threads 5B to couple the cap to the actuating stem 5. The lower part of the actuating stem 5 passes into the cylindrical rotor block 6 via the centrally located oval-shaped tunnel of the rotor block 6. The oval-shaped portion 5C of the actuating stem 5 rests in the tunnel of the rotor block 6, as shown in FIG. 2B.

A cross-sectional width of the middle oval-shaped portion 5C of the actuating stem 5 is greater than a cross-sectional width of the round portions 5A and 5D. A shape of the centrally located tunnel of the rotor block 6 may correspond to the cross-section of the middle portion 5C of the actuating stem 5, to facilitate rotation of the rotor block 6 in response to rotation of the actuating stem 5. Although FIG. 1A illustrates the cross-sections of the portions 5A, 5C and 5D as being round, oval-shaped and round, in other embodiments the portions of the actuating stem 5 may have other cross-sectional shapes, such as triangles, squares, rectangles, pentagons, hexagons, etc.

As mentioned above, the cap includes an upper portion 2 and a lower portion 3. Three channels extend through the upper and lower portions 2 and 3. The two outer channels 2A are injection ports, and the center channel 2B allows the actuating stem 5 to pass though the cap. The lower portion 3 may be coupled to the lower half of the device 100 by twisting male screw threads of the lower portion onto female threads 7 of the device housing 8.

The cap also includes two plugs 2C, which facilitate maintaining the sterility of the device 100 throughout the whole process of tissue disaggregation by blocking internal compartments of the device 100 from the outside environment. The plugs 2C may include any suitable material for plugging the channels 2A, such as silicone, plastic, etc. The upper and lower portions 2 and 3 of the cap may include a solid material such as plastic, metal, etc. In some embodiments, the nut 1, the spring 4, the actuating stem 5 and the cylindrical rotor block 6 are made of metal materials.

The device housing 8 includes the female screw threads 7 on an inner surface of the opening of the housing 8, and side blocks 9 and 10 for stabilization of the housing 8 in the base 16 (as shown in FIGS. 2A and 2B) during motorized or manual tissue disaggregation. The housing 8 also includes a recess space for positioning the grater 12, and female screw threads 11 for coupling with male threads 15 on the collection vessel 13.

A cylindrical column 14 is positioned in the center of the collection vessel 13, and the column 14 has a hollow center for receiving the lower round portion 5D of the actuating stem 5. The centrally located column 14 directly supports the grater 12 during the trituration process, and also holds the lower round portion 5D of the actuating stem 5 to effectively inhibit (e.g., prevent) bending of the grater 12 at the center and wobbling of the whole device 100 during the triturating process. This three-point stabilization design for the actuating stem 5 (e.g., at the cap portions 2 and 3, at the grater 12, and at the supporting hollowed column 14) is mechanically sound and facilitates stable and efficient tissue trituration. FIG. 1B illustrates an assembled lower half of the device 100, which may be made mostly of plastic so the lower half assembly may be a one-time use disposable item to prevent cross-contamination.

FIG. 2A is an exploded sectional view of the device 100, while FIG. 2B is a sectional view of the assembled device 100. The grater 12 may be detachable as shown in FIG. 2A. Alternatively, the grater 12 may be embedded into the lower part of the housing 8, integral with the housing 8, etc. Similarly, the collection vessel 13 may be fused into the housing 8, etc. The base 16 may have any size and/or shape that is suitable for stabilizing the device 100 during tissue processing.

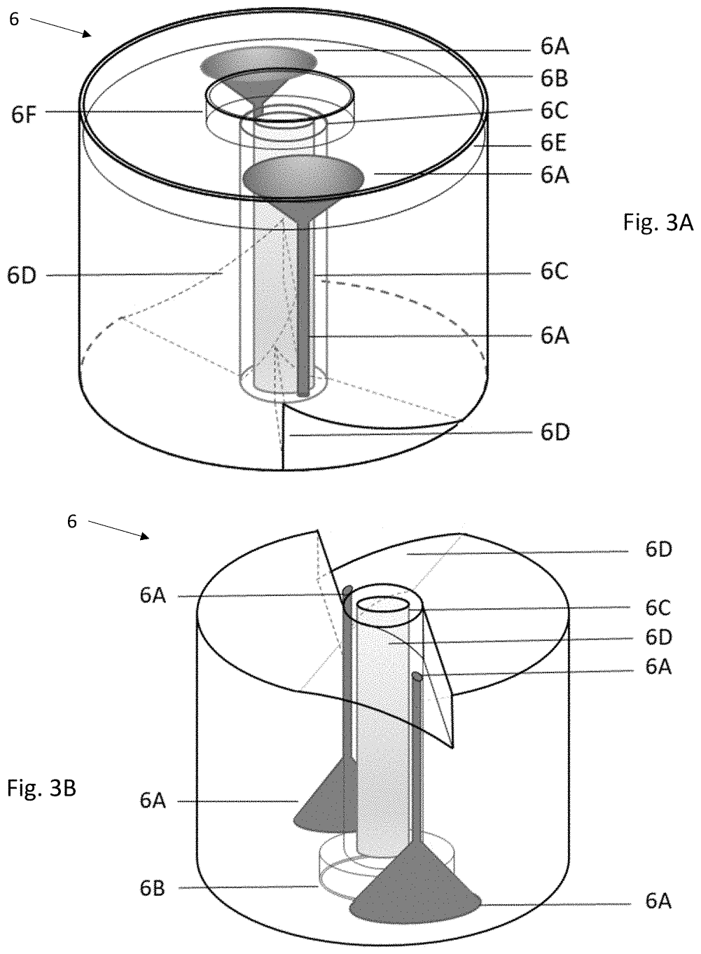

FIG. 3A illustrates a transparent view of the cylindrical rotor block 6. The block 6 may be made of metal, with a center tunneled out for receiving the actuating stem 5. As mentioned above, the stem may be round and narrower at the end portions 5A and 5D, with an oval cross-sectional shape in the middle portion 5C. Using a heavy and solid piece of metal for the rotor block 6 increases the stability of rotation of the rotor block 6, thus making the trituration process more effective, as compared to lighter and thinner sheet blades used in prior art devices. The oval-shaped portion 5C of the actuating stem 5 is inserted into the oval-shaped tunnel 6C of the rotor block 6, which causes rotation of the rotor block 6 when the actuating stem is rotated via a motor, a hand crank, etc.

As shown in FIG. 3A, a periphery 6E of the top of the rotor block 6 is raised, as well as a wall 6F of the circular recess 6B. There are two funnels 6A that connect upper and lower surfaces of the rotor block 6. These two funnels 6A may be aligned with the outer channels 2A on the cap, to allow the addition of a buffer material into the spaces 6D where tissue specimens are being triturated. However, the funnels 6A of the rotor block 6 are not required to be aligned with the outer funnels 2A of the cap, because the two circular walls 6E and 6F are raised high enough to allow the added buffer material to freely flow into the two funnels 6A after being deposited through the outer funnels 2A of the cap. In the center of the rotor block 6, the circular recess 6B allows seating of the spring 4, when the stem 5 is in place and the cap is tightened onto the housing 8. Therefore, the spring 4 can exert the necessary pressing force onto the rotor block 6, to further reinforce the stability and effectiveness of the trituration process.

FIG. 3B illustrates an upside down view of the rotor block 6. The exits of the funnels 6A are located at two diametrically opposed recesses 6D. The two recesses 6D may function as the rotor blades for the rotor block 6. Each recess/rotor blade 6D extends tangentially from the centrally located, oval-shaped tunnel, extending outwards and making a vertical cut in the rotor block 6D until the recess/rotor blade 6D reaches a perimeter of the rotor block 6. Next, the cut of the recess/rotor blade 6D folds, bows up and extends clockwise for a specified distance around a circle (e.g., the curved portion of the rotor blade). The recess/rotor blade 6D ends with a flat surface on the rotor 6 (e.g., the flat portion of the rotor blade). The flat surface may be matted for an increased trituration effect. Tissue specimens may be placed in the bowed, curved recess 6D. During the tissue disaggregation process, tissue specimens will be gradually dispersed under the flat portions of the rotor blade for trituration against the grater 12.

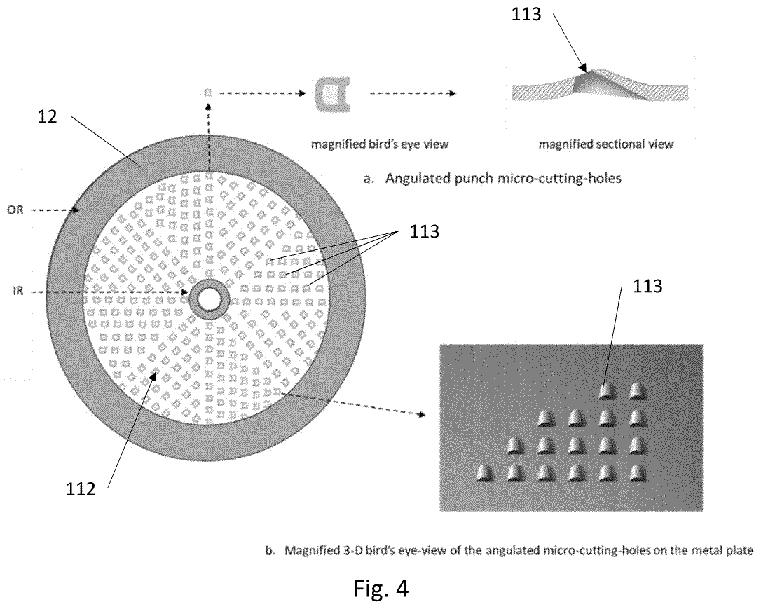

Opposite to trituration surface of the rotor 6 is the trituration grater 12, which may include a mesh screen/sheet 112 as shown in FIG. 4. The example mesh screen design of FIG. 4 includes groups of radially located, angled micro-cutting holes 113. FIG. 4 illustrates both a magnified top view and a magnified sectional view of a single micro-cutting hole 113. The raised micro-cutting holes 113 may be stamped, punched out, etc., to project outwardly in a range between approximately fifteen to sixty degrees with respect to the metal sheet 112 of the trituration grater 12, with the cutting blades facing oncoming tissue specimens during the trituration process. In some embodiments, the entry angle of the micro-cutting holes 113 may be anywhere in the range from zero degrees (e.g., a flat hole), to ninety degrees (e.g., a vertically raised hole), although cutting efficiency may be reduced outside of the range between fifteen and sixty degrees.

The sheet 112 have a thickness in a range from about fifty to about five hundred microns in order to increase sharpness of the blades for processing samples, although other embodiments may have different thicknesses depending on the product required, the metal(s) used, the size of the device, etc. One advantage of the stamping or punching method is that the micro-cutting edges may have burrs, which enhance the cutting ability of the micro-cutting-holes 113. The micro-cutting-holes 113 may have a diameter in a range from about forty microns to about five hundred microns, although other diameters may also be used. The size of the micro-cutting holes 113 may allow the tissue disaggregation method to process tissue specimens in a range from single cells up to cell aggregates having a few thousand cells, etc.

This trituration grater 12 may be wrapped at the inner and outer edges with metal rims for increased rigidity and strength. The inner rim (IR) of the grater 12 may be supported by the column 14 in the collection vessel 13. The outer rim (OR) of the grater 12 may be held firmly by a solid grip between the thick device housing 8 and the collection vessel 13. As a result, the grater 12 may not be crushed, deformed, etc., during tissue processing.

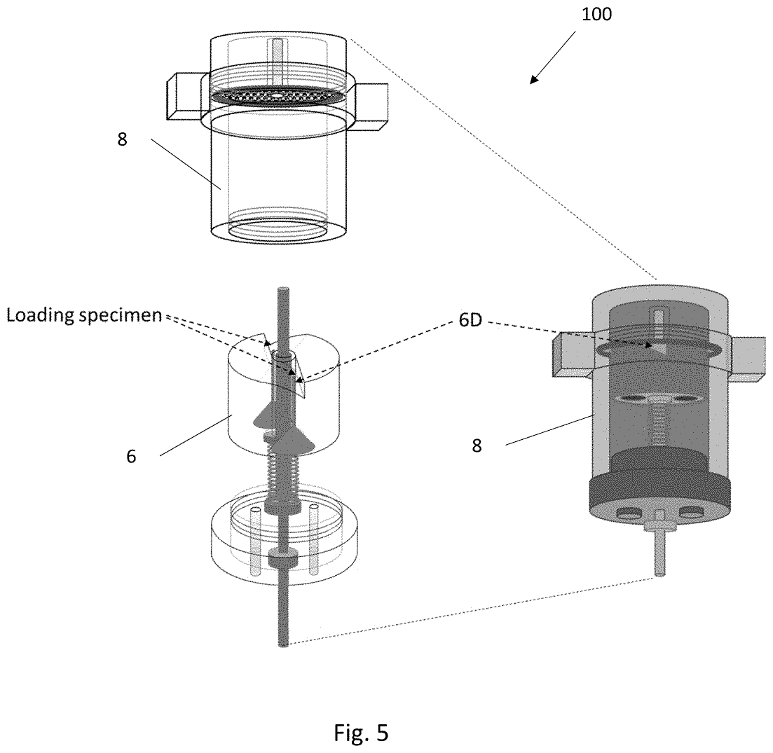

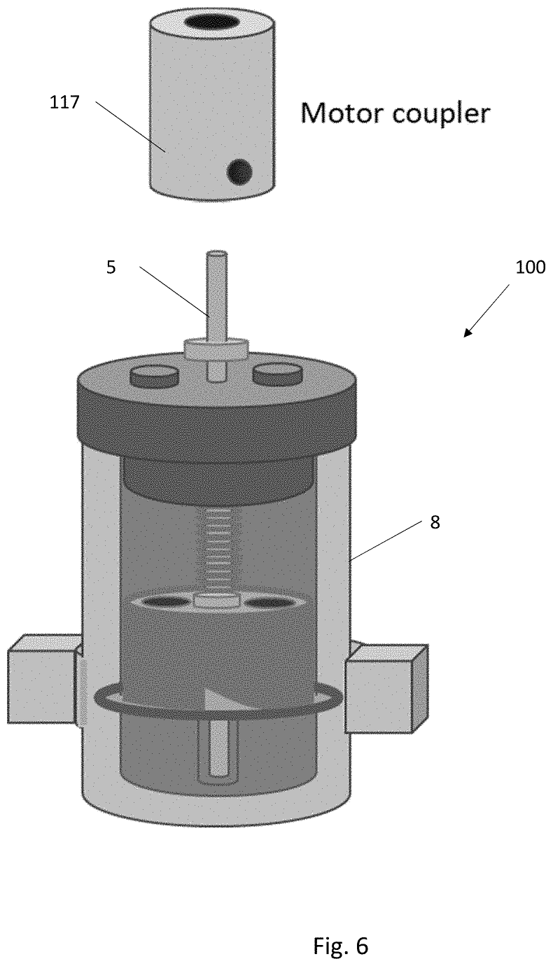

FIG. 5 illustrates example locations for loading tissue specimens into the device 100. For example, in order to load a tissue specimen into the recesses 6D in the rotor block 6, the upper half of the device 100 may be inverted so the rotor block 6 is upside down as shown in FIG. 5. Solid or semi-solid specimens may be loaded onto the recesses 6D. Next, the inverted upper half of the device 100 may be inserted into the inverted device housing 8, as shown in the assembled device portion of FIG. 5. Once the device 100 is assembled with the specimens in recesses 6D, the device is turned over to the normal upright position, as shown in FIG. 6.

FIG. 6 illustrates a motor coupler 117 that may be connected with the actuating stem 5 to provide motorized rotation of the stem 5 and the rotor block 6. The motor coupler 117 may include any suitable coupling element for connecting a motor shaft, etc., with the stem 5. Alternatively, a hand crank may be coupled to the actuating stem 5 to allow for manual rotation of the actuating stem 5 and the rotor block 6. When desired, the whole tissue processing operation could be performed on an operating room side table, making it easier for surgeons to keep the process sterile.

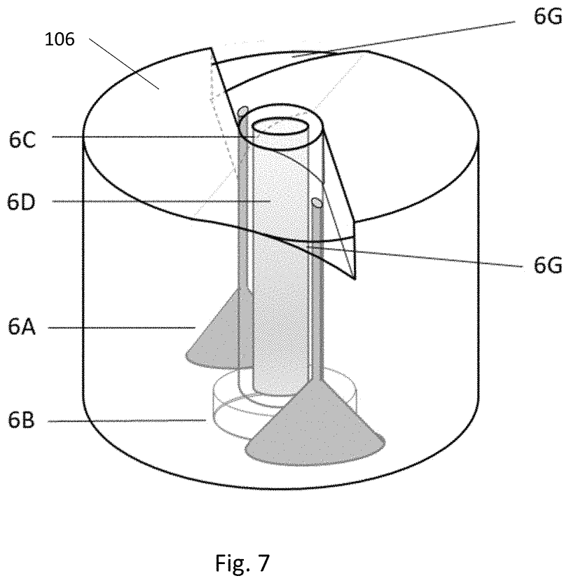

FIG. 7 illustrates a rotor block 106 including a side wall 6G along a portion of the recess 6D, according to another example embodiment. As shown in FIG. 7, the side wall 6G is located along a perimeter of the rotor block 106, adjacent the deeper portion of the recess 5D. Therefore, semi-liquid tissue specimens may be loaded into the recess 6D without leaking out of the recess 6D.

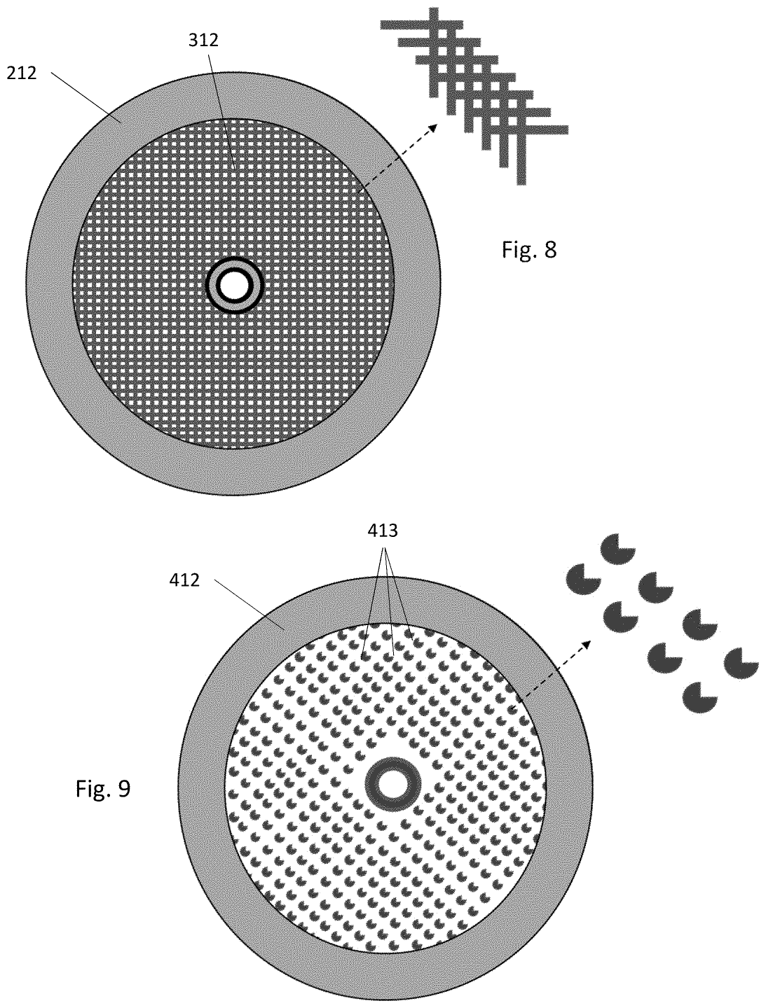

FIGS. 8 and 9 illustrate alternative designs for trituration graters 212 and 412, according to additional example embodiments. As described above, one design for the mesh sheet 112 of the grater 12 in FIG. 4 includes radially located, angulated micro-cutting holes 113. This design may be ideal for tissues specimens such as scalp tissue, periosteum tissue, tendinous tissue, etc. Alternatively, FIG. 8 illustrates a trituration grater 212 where the mesh screen 312 includes interwoven wire mesh. This screen design may be preferred when the tissue specimen to be processed is an atrial appendage tissue, umbilical cord Wharton's jelly, etc.

FIG. 9 illustrates another alternative trituration grater 412, where the cutting openings 413 (e.g., pores) include laser etched configurations of various geometric shapes. In the example embodiments of FIGS. 8 and 9, the thickness of the mesh screen may be in a range from about fifty to five hundred microns, depending on the need and the metal strength. The pore size may be in a range from about forty to five hundred microns, depending on the type of tissue that is being processed, a size of the device, etc. In other embodiments, the mesh screen may have a smaller or greater thickness, the pores may have smaller or greater sizes, etc.

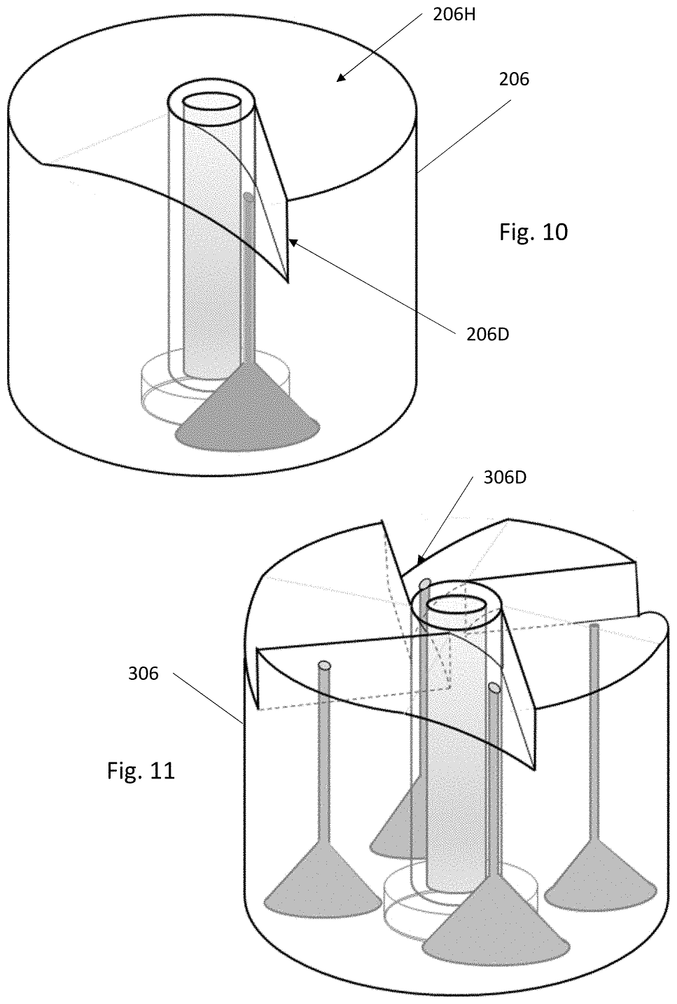

Although FIG. 3B illustrated the rotor block 6 as having two recesses 6D, in other embodiments the rotor block may have more or less than two recesses/rotor blades. For example, FIG. 10 illustrates a rotor block 206 having only one recess/rotor blade 206D, which may be deeper than the recesses 6D of the rotor block 6. Additionally, the recess/rotor blade 2-66D of the rotor block 206 of FIG. 10 may provide a flat surface 206H having an increased length for trituration as compared to the rotor blades of the rotor block 6. Alternatively, FIG. 11 illustrates a rotor block 306 including four recesses/rotor blades 306D. In other embodiments, the rotor block may have three rotor blades, five rotor blades, or any number of rotor blades up to even one hundred or more.

In yet another embodiment, the rotor blade may protrude from the surface of the rotor block, instead of recessing into the surface of the rotor block. For example, the rotor block may include one protruding rotor blade, two protruding rotor blades, etc., up to even twelve protruding rotor blades or more, which are suitable to facilitate trituration by pressing the tissue samples against the trituration grater 12.

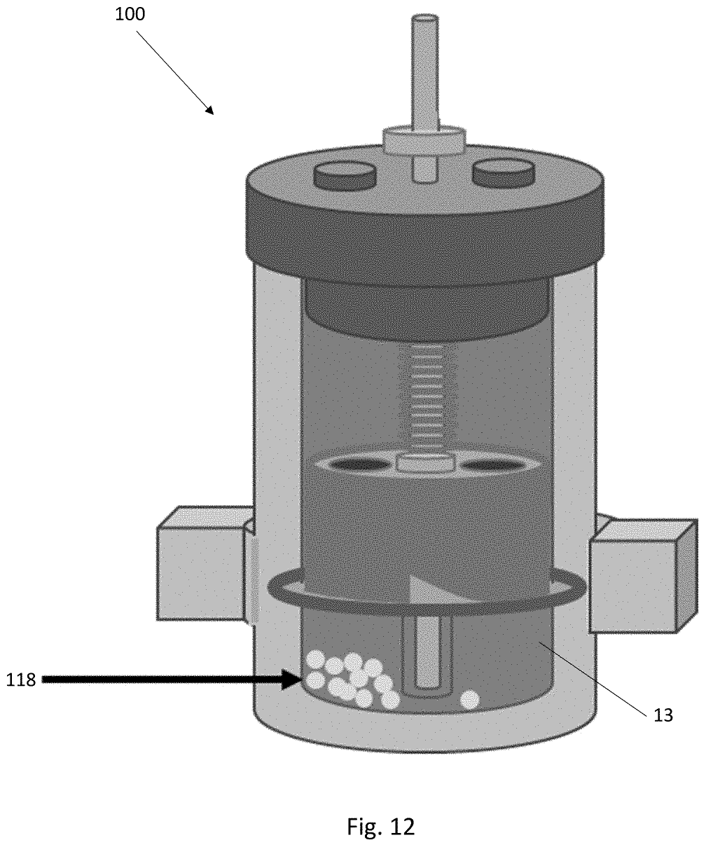

FIG. 12 illustrates the device 100 with bead mill 118 located in the collection vessel 13. The bead mill 118 may be used for further cell processing, such as when single cell suspensions are more desirable. FIG. 12 illustrates twelve stainless steel beads located in the collection vessel 13, each having a diameter of two millimeters. In other embodiments, more or less beads may be used, beads of other materials may be used, beads having smaller or larger diameters may be used, etc.

At the end of the collection phase of the tissue processing, if single cells are desired instead of cell aggregates, a user may shake the apparatus a specified number of times (e.g., about sixty to one hundred times over twenty to thirty seconds, etc.). This may cause the cell aggregates to break apart to become a single cell suspension. The whole process may be performed under a sealed, sterile condition. The single cell suspension may then undergo filtration for the removal of larger debris. The filtrated product is then pelleted and resuspended for further applications.

In yet another embodiment, enzymatic digestion may be used to obtain a single cell suspension. The collection vessel 13 may be transformed into an incubator by adding enzymes such as collagenase (or any other suitable tissue disaggregation chemical agent) in the vessel 13 prior to initiation of the trituration. Later, with the processed cell aggregates and the buffer material coming down into the vessel, the whole system could be placed into an incubator (e.g., at 37°, etc.) for the enzyme to take full effect, while keeping the whole tissue processing in an enclosed and sterile manner. After the incubation is finished, the enzyme activity may be terminated. The cells may then be washed, pelleted, and resuspended for the next step in application.

The trituration device 100 may be used for any suitable tissue trituration, such as preparation of micrografts from hair follicle bearing scalp tissue. For example, two punch biopsies (e.g., two mm in diameter, three mm in diameter, five mm in diameter, etc.), may be performed under local anesthesia to harvest two pieces of hair follicle bearing tissues from the posterior aspect of the patient's scalp. The depth of the biopsy may be about seven to eight mm. The scalp wounds are closed using cat gut sutures.

On the specimens, the hair is trimmed along with the epidermal skin layer using a blade, because the epidermal skin cells may form cysts once imbedded back into the dermal and subcutaneous layers. The tissue is further sliced into 1.5 to two mm wide pieces longitudinally, with two drops of PBS to prevent tissue desiccation. This is performed in a petri dish under sterile conditions, preferably in a laminal flow hood or in the operating room. Afterwards, the tissue pieces are weighed.

Next, the tissue pieces are loaded onto the recesses 6D on the inverted rotor block 6, as illustrated in FIG. 6. The two plugs 2C are placed in the outer channels 2A of the cap, as shown in FIGS. 1A and 1B. This is followed by insertion of the inverted, loaded upper half of the device 100 into the housing 8 of the inverted lower half of the device, as shown in FIG. 5. Once the device 100 is assembled, it is turned around to the normal upright position, as shown in FIG. 6.

With a commercial motor coupler 117, the invented device may be connected to a low speed motor. Motorized tissue disaggregation is performed for a specified time period at a specified speed (e.g., about three minutes at about sixty revolutions per minute (RPM), etc.). The motor may be temporarily stopped, and PBS (e.g., about two milliliters) is injected into one of the outer channels 2A of the cap. The PBS will flow down the funnels 2A into the recesses 6D, where the tissue is being triturated.

The motor is restarted for more tissue disaggregation (e.g., about two more minutes, etc.), and the process of stopping to add PBS may be repeated as many times as desired. The device 100 is then detached from the motor and shaken to allow the fluid in the collection vessel 13 to wash off any pasty tissue that is attached to the undersurface of the grater 12.

The collection vessel 13 is then detached from the housing 8, and the bottom of the grater 12 is flushed with PBS (e.g., about one cc of PBS, etc.), if any residual tissue remains attached to the under surface of the grater 12. The cell aggregates in the tissue collection vessel 13 are then collected, the cap is untwisted and the rotor block 6 is removed, and the tissue pieces left on the grater 12 are weighed (e.g., to determine leftover tissue weight).

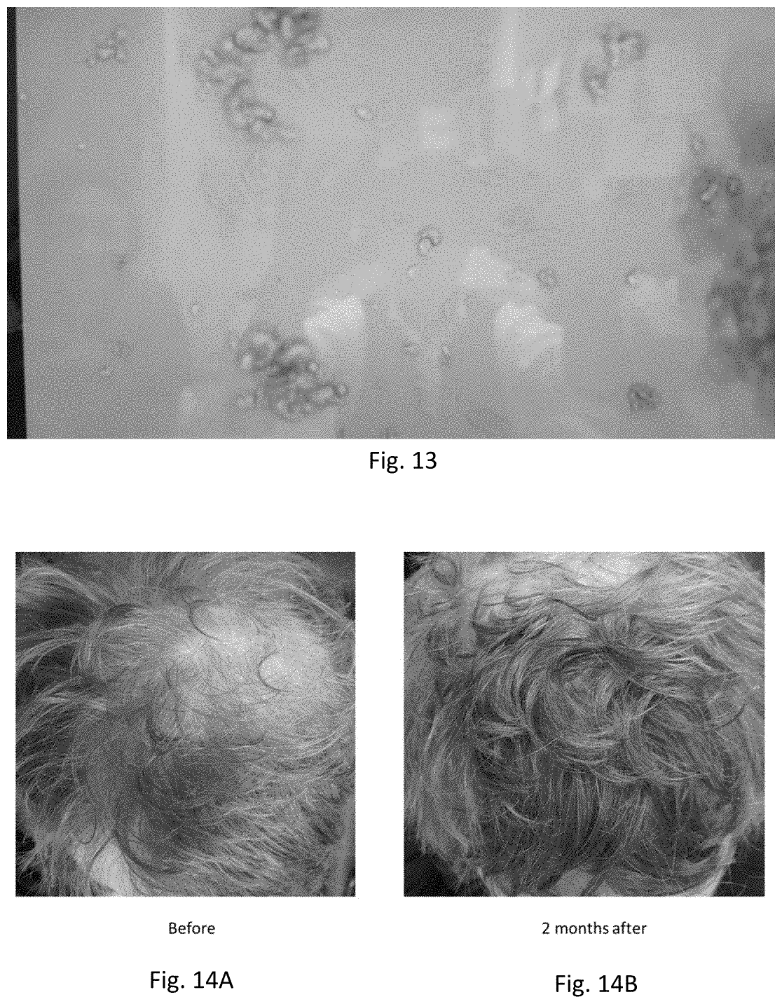

The efficiency of tissue processing is demonstrated by the amount of tissue disaggregated (e.g., total original weight minus leftover tissue weight) in the collection vessel 13, divided by the total amount of original tissue weight. Experimental data using the device 100 showed a recovery rate of about 40%. It may be desirable to have an efficiency of at least 10% of recovered disaggregated tissue, so the device 100 provides much higher efficiency that prior art devices that only had an efficiency of 0.076%. When the cell aggregates are observed under a microscope, most of the cells have the form shown in FIG. 13 under 100× magnification.

Because it is not possible to obtain a precise count of the cells when they are in aggregates, the number of cells may be counted by subjecting half of the harvested cell aggregates to bead mill disaggregation treatment in a twelve cc test tube using three mm stainless steel beads for twenty seconds of shaking by hand (e.g., sixty times), followed by hemocytometer counting. In one experiment, the cell number totaled at 0.83×10⁶ live cells, so the total yield was 1.66×10⁶ cells for the two whole pieces of biopsied scalp tissue.

The untouched 50% of the cell aggregates were put into four cc of PBS for grafting injection. The bald area on the patient's parietal region was marked and prepared. A total of twenty points spaced out two cm from each other were injected with a 25G needle, without local anesthesia. The injection was at the subcutaneous layer at four to six mm in depth, with 0.2 cc for each point. FIGS. 14A and 14B show the result of this treatment for a 35-year-old male patient with male pattern hair loss. FIG. 14A is a before image and FIG. 14B is an image taken two months after the treatment.

As described above, the inventors of the present application recognized that the device 100 may provide improved efficiency for hair follicle tissue disaggregation and improved hair regeneration results compared to prior devices, and the device 100 may be used for more efficient processing of other tissues in both clinical and laboratory settings. The inventors found that in some cases cutting or scissoring coupled with triturating could obtain finely disaggregated micrografts, where the trituration device 100 may provide a reliable, cost-effective and much higher tissue disaggregation efficiency than previously known tissue disaggregation devices.

Another example use for the device 100 is preparation of cardiac atrial appendage tissue for tissue disaggregation. Heart disease is one of the major health problems worldwide. In the United States alone, the cost for cardiac health care is more than $400 billion a year. Among the various conditions, myocardial infarct (MI) is a more significant issue because once it occurs, many patients undergo cardiac failure within a few years. And the mortality rate is high, despite aggressive treatments. One of the most important reasons is that the cardiac muscle tissue fails to restore its forms and functionalities, due to disorganized tissue repair after the ischemic event. In the past ten years, cell regenerative therapy has generated high hopes in this regard, as described by Smits A M, van Laake L W, den Ouden K, et al. Human cardiomyocyte progenitor cell transplantation preserves long-term function of the infarcted mouse myocardium. Cardiovasc. Res. 2009; 83: 527-535.

The available data show that the infarcted muscles are restored and the ventricular functions return to normal after cell treatments, as described by Fanton Y, Robic B, Rummens J L, Daniels A, Windmolders S, Willems L, Jamaer L, Dubois J, Bijnens E, Heuts N, Notelaers, K, Paesen R, Ameloot M, Mees U, Bito V, Declercq J, Hensen K, Koninckx R and Hendrikx M. Cardiac atrial appendage stem cells engraft and differentiate into cardiomyocytes in vivo: A new tool for cardiac repair after MI. Int. J Cardiol. Volume 201, p. 10-19, 15 Dec. 2015. DOI: https://doi.org/10.1016/j.ijcard.2015.07.066. The cell transplantation treatment could be done with autologous fat mesenchymal stem cells, cultured cardiac muscular progenitor cells, or micrografts generated from atrial appendage biopsy specimens, as described by Nummi A, Nieminen T, Pätilä T, Lampinen M, Lehtinen M, Kivistö S, Holmström M, Wilkman E, Teittinen K, Laine M, Sinisalo J, Kupari M, Kankuri E, Juvonen T, Vento A, Suojaranta R, and Harjula A. Epicardial delivery of autologous atrial appendage micrografts during coronary artery bypass surgery—safety and feasibility study. Pilot and Feasibility Studies. (2017) 3:74. DOI 10.1186/s40814-017-0217-9.

The micrografts are obtained by taking specimens from the atrial appendages, and the micrografts are available within 30 minutes from the time of biopsy, thus making urgent cell therapy for MI a real possibility. And the results from both animal models and human cases so far show great results. On the other hand, cell therapy with expanded cells from cell culture takes weeks to prepare, which drastically increases the waiting time, and the therapeutic results are therefore limited. The micrografts used for cell therapy are therefore preferred by most practitioners. Described below is an example for obtaining the micrografts.

First, porcine cardiac atrial appendage tissues are obtained from a local slaughterhouse within four hours, etc., of a sacrifice. The tissues are kept on ice and small biopsies are done to obtain one gram, etc., of specimens. The biopsy tissues are sliced into two mm wide pieces longitudinally, with two drops of PBS to prevent tissue desiccation. This is performed in a petri dish under sterile condition, in a laminal flow hood. Afterwards, the tissue pieces are weighed.

Next, the tissue pieces are loaded onto the recesses 6D on the inverted rotor block 6, and the two plugs 2C are placed in the outer channels 2A of the cap. The inverted, loaded upper half of the device 100 is inserted into the housing 8 of the inverted lower half of the device 100. Once the device is assembled, it is turned around to the normal upright position, as shown in FIG. 6.

With a motor coupler 117, the device 100 is connected to a low speed motor. Motorized tissue disaggregation is performed for three minutes at about 60 RPM. The motor is temporarily stopped, and two milliliters of PBS are injected into one of the channels 2A. The PBS will flow down the funnels into the recesses 6D, where the tissue is being triturated. The motor is then restarted for two more minutes of tissue disaggregation, and this process may be repeated as many times as desired.

The device 100 is then detached from the motor and shaken to allow the fluid in the collection vessel 13 to wash off any pasty tissue attached to the undersurface of the grater 12. The collection vessel 13 is detached from the housing, and the bottom of the grater 12 is flushed with one cc of PBS if any residual tissue remains attached to the under surface of the grater 12.

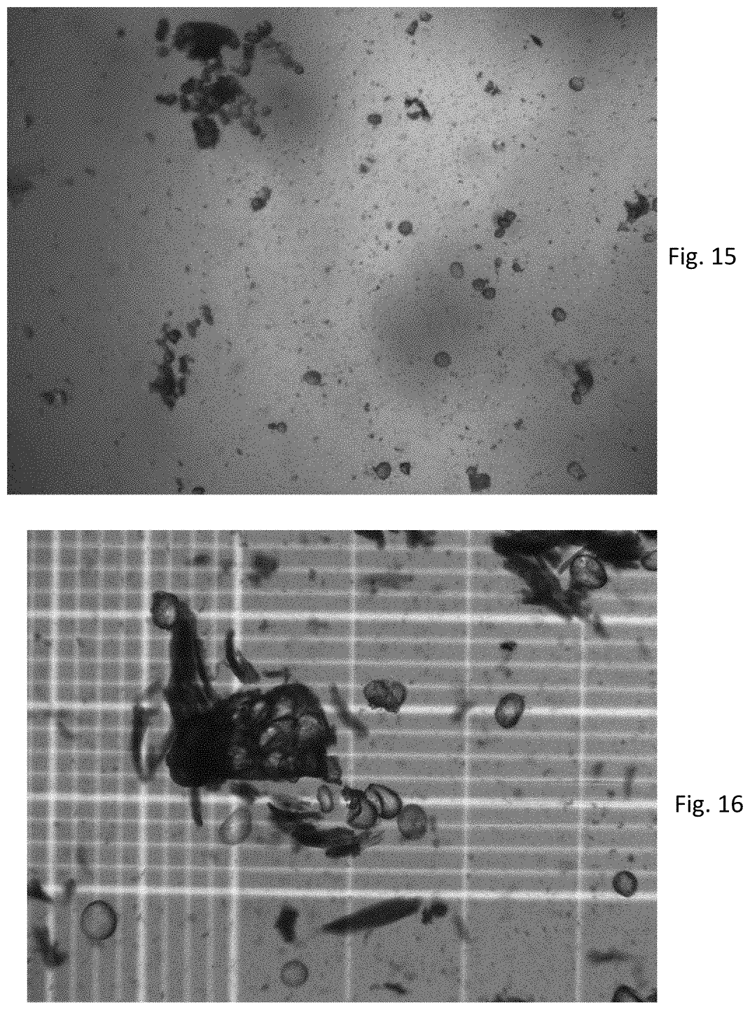

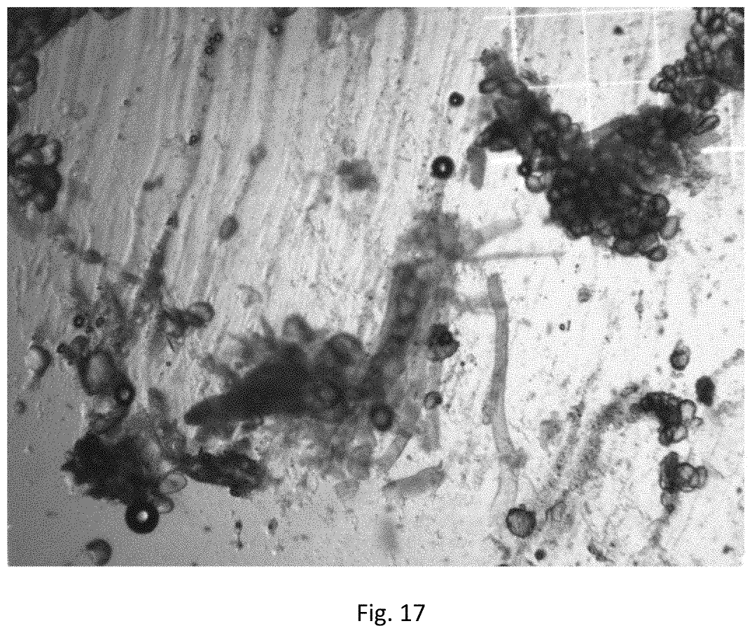

The cell aggregates in the tissue collection vessel 13 are collected, the cap is untwisted and the rotor block 6 is removed. Any tissue pieces left on the grater 12 are weighed to determine the weight of leftover tissue. The efficiency of tissue processing is determined by the amount of tissue disaggregated (e.g., the total original weight minus leftover tissue weight) in the collection vessel 13, divided by the total amount of original tissue weight. One experiment showed a recovery rate of about 63%. The cell aggregates may be observed under a microscope. Most of the cells may be in the form of cell aggregates and single cells as shown in FIG. 15 at 40× magnification, and broken muscle fibers as shown in FIG. 16 at 100× magnification with the fiber-shaped tissue in blue.

The mix of cell aggregates and single cells may now be used for a primary tissue culture for the characterization of the cardiac atrial appendage progenitor cells. Because it is impossible to obtain a precise count of the cells when they are in aggregates, the number of cells in the experiment was counted by subjecting half of the harvested cell aggregates to bead mill disaggregation treatment in a 12 cc test tube using three mm stainless steel beads for 20 seconds of shaking by hand (e.g., 60 times), followed by hemocytometer counting. In the experiment, the cell number totaled at 1.6×10⁶ live cells, so the total yield 3.2×10⁶ cells for the whole lot of one gram of biopsied atrial appendage tissue. Almost all the muscle fibers are stainable with trypan blue. These singular cells could also be used for primary tissue culture. The untouched 50% of the cell aggregates may now be used for intramyocardial injection for the treatment of experimental infarction. The injection needle could be 25G, 26G, 27G, etc.

As another example, the device 100 may be used for preparation of rib periosteum for tissue disaggregation. In dental and orthopedic fields, bone regeneration is one of the most important issues. The deficiency in alveolar ridge, for example, could be caused by extraction of teeth. This deficiency in turn, affects the suitability of the subsequent implant surgery. Similarly, bone fractures, without proper blood supply and osteoplastic cell activity, will be difficult to heal properly. Cell based therapy, is therefore gaining more and more popularity in both fields, as described by Sancho F M, Leira Y, Orlandi M, Buti J, Giannobile W V, and D'Aiuto F. Cell-based Therapies for Alveolar Bone and Periodontal Regeneration: Concise Review. Stem Cells Translational Med. 2019:8; 1286-1295, in the hope of helping tissue regeneration of the dental bones or healing of bone fractures. This device 100 could be used to prepare cell aggregates as the micrografts in both fields.

For example, a periosteum biopsy may be performed in the mastoid region, utilizing a post-auricular incision, under local anesthesia. The periosteum tissue is sliced into 5 mm×2 mm pieces, with two drops of PBS to prevent tissue desiccation in a petri dish under sterile conditions, preferably in a laminal flow hood or in the operating room. Afterwards, the tissue pieces are weighed.

Next, the tissue pieces are loaded onto the recesses 6D on the inverted rotor block 6, and the two plugs 2C are in placed in the channels 2A of the cap. This is followed by insertion of the inverted, loaded upper half of the device 100 into the housing 8 of the inverted lower half of the device 100. Once the device 100 is assembled, it is turned around to the normal upright position, as shown in FIG. 6.

With a commercial motor coupler 117, the device 100 is connected to a low speed motor. Motorized tissue disaggregation is performed for three minutes at about 60 RPM. The motor is temporarily stopped, and two milliliters of PBS are injected into one of the channels 2A. The PBS will flow down the funnels into the recesses 6D, where the tissue is being triturated. The motor is then restarted for two minutes of tissue disaggregation. The above process may be repeated as many times as desired.

The device 100 is then detached from the motor and shaken to allow the fluid in the collection vessel 13 to wash off any pasty tissue attached to the under surface of the grater 12. The collection vessel 13 is detached from the housing 8, and the bottom of the grater 12 is flushed with one cc of PBS if any residual tissue remains attached to the under surface of the grater 12.

The cell aggregates in the tissue collection vessel are collected, the cap is untwisted, and the rotor block 6 is removed. The tissue pieces left on the grater 12 weighed to determine a weight of the leftover tissue. The efficiency of tissue processing is determined by the amount of tissue disaggregated (e.g., the total original weight minus leftover tissue weight) in the collection vessel 13, divided by the total amount of original tissue weight. In one experiment, the process showed a recovery rate of about 43%. The cell aggregates may be observed under a microscope. Most of the cells may be in forms as shown in FIG. 17 at 100× magnification. The cell aggregates can now be used for the intended applications.

Although the above processes are described as using specified lengths, time periods, volumes, etc., other embodiments may use other parameters without departing from the scope of the present disclosure.

According to another example embodiment, a method of disaggregating tissue using a trituration device is disclosed. The device includes a device housing, a rotor block comprising a solid cylinder including a central channel and at least one recess that defines a rotor blade, a trituration grater including multiple angled cutting holes, a collection vessel to collect disaggregated tissue, and an actuating stem.

The method includes positioning a tissue specimen within the at least one recess of the rotor block, inserting the rotor block into the device housing to contact the trituration grater, while the tissue specimen remains in the at least one recess of the rotor block, inserting the actuating stem through the central channel of the rotor block, and rotating the actuating stem using a motor or crank to rotate the rotor block and press the tissue specimen against the trituration grater to receive disaggregated tissue in the collection vessel.

The device may include a cap having at least one side channel and at least one plug received in the at least one side channel. The method may include coupling the cap to an end of the device housing opposite the collection vessel, removing the at least one plug from the at least one side channel, injecting a buffer material though the at least one side channel to facilitate trituration of the tissue specimen, and replacing the at least one plug after injection to maintain sterility of the device during operation.

In some embodiments, the method may include placing multiple beads in the collection vessel and shaking the collection vessel after receiving the disaggregated tissue to facilitate breaking apart of the disaggregated tissue, and/or placing one or more tissue disaggregation agents in the collection vessel to provide tissue disaggregation for tissue specimens received in the collection vessel.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. In addition, advantages and improvements that may be achieved with one or more exemplary embodiments of the present disclosure are provided for purposes of illustration only and do not limit the scope of the present disclosure, as exemplary embodiments disclosed herein may provide all or none of the above mentioned advantages and improvements and still fall within the scope of the present disclosure.

Specific dimensions, specific materials, and/or specific shapes disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters. For example, the terms “generally”, “about”, and “substantially” may be used herein to mean within manufacturing tolerances.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

The foregoing description of the embodiments has been provided for purposes of illustration and description. It is not intended to be exhaustive or to limit the disclosure. Individual elements, intended or stated uses, or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A trituration device for tissue disaggregation, the device comprising:
a device housing, the device housing comprising a hollow cylinder defining an opening extending from a first end of the device housing to a second end of the device housing;
a rotor block positioned in the opening of the device housing, the rotor block comprising a solid cylinder including a central channel and at least one surface that defines a rotor blade;
a trituration grater positioned within the opening of the device housing to contact the rotor block, the trituration grater including multiple angled cutting holes;
a collection vessel positioned at the second end of the device housing to collect disaggregated tissue of a tissue specimen; and
an actuating stem extending through the central channel of the rotor block to rotate the rotor block in response to rotation of the actuating stem.

2. The device of claim 1, wherein:
the actuating stem includes a first end, a second end opposite the first end, and a middle portion defined between the first end and the second end;
a cross-sectional width of the middle portion is greater than a cross-sectional width of the first end and the second end;
a shape of the central channel of the rotor block corresponds to a shape of the cross-section of the middle portion of the actuating stem, to facilitate the rotation of the rotor block in response to rotation of the actuating stem;
the collection vessel includes a central column defining an opening; and
the second end of the actuating stem is received in the opening of the central column.

3. The device of claim 2, wherein the shape of the cross-section of the middle portion of the actuating stem is one of an oval, a triangle and a square.

4. The device of claim 2, wherein the actuating stem comprises a coupler at the first end, the coupler adapted to connect the actuating stem to a motor for motorized operation or to a crank for manual operation.

5. The device of claim 1, further comprising a cap coupled to the first end of the device housing, wherein the cap includes:
a central channel for receiving the actuating stem;
at least one side channel for injection of a buffer material to facilitate trituration of the tissue specimen; and
at least one plug received in the at least one side channel to maintain sterility of the device during operation.

6. The device of claim 5, further comprising a spring positioned between the cap and the rotor block to exert a force on the rotor block in a direction of the trituration grater.

7. The device of claim 1, wherein the at least one surface of the rotor block that defines the rotor blade includes at least one surface that protrudes from the rotor block to contact the triturating grater.

8. The device of claim 1, wherein the at least one surface of the rotor block that defines the rotor blade includes at least one recess for receiving the tissue specimen, the at least one recess defining the rotor blade.

9. The device of claim 8, wherein:
the at least one recess of the rotor block comprises two recesses that define two rotor blades;
the rotor block includes a surface opposite the two recesses; and
the rotor block includes two funnels to facilitate passage of a buffer material to the two recesses from the surface opposite the two recesses.

10. The device of claim 8, wherein each rotor blade includes a surface perpendicular to the trituration grater, a surface parallel to the trituration grater, and a curved portion between the perpendicular surface and the parallel surface.

11. The device of claim 8, wherein the rotor block includes a sidewall disposed along an outer side of each recess to retain semi-liquid tissue specimens in each recess.

12. The device of claim 1, wherein:
the multiple angled cutting holes are positioned at an outward angle of between fifteen and sixty degrees with respect to a plane of the trituration grater;
the trituration grater has a thickness of about fifty to about five hundred microns; and
a diameter of each of the multiple angled cutting holes is about forty to about five hundred microns.

13. The device of claim 1, wherein:
the collection vessel is integrated with the device housing or is coupled to the device housing via screw threads; and
the collection vessel includes multiple beads to facilitate breaking apart of tissue aggregates received in the collection vessel, in response to shaking of the collection vessel.

14. The device of claim 1, wherein the collection vessel includes one or more tissue disaggregation agents to provide tissue disaggregation for tissue specimens received in the collection vessel.

15. The device of claim 1, further comprising a support base for stabilizing the device during operation, wherein the device housing includes two side blocks, and the support base is adapted to receive the side blocks to stabilize the device during operation.

16. A trituration device for tissue disaggregation, the device comprising:
a device housing;
a rotor block positioned in the device housing, the rotor block including a central channel and at least one rotor blade;
a trituration grater positioned within the device housing to contact the rotor block, the trituration grater including multiple angled cutting holes;
a collection vessel positioned at an end of the device housing to collect disaggregated tissue of a tissue specimen; and
an actuating stem including a first end, a second end opposite the first end, and a middle portion defined between the first end and the second end, wherein:
a cross-sectional width of the middle portion is greater than a cross-sectional width of the first end and the second end;
the actuating stem extends through the central channel of the rotor block, and a shape of the central channel of the rotor block corresponds to a shape of the cross-section of the middle portion of the actuating stem to facilitate rotation of the rotor block in response to rotation of the actuating stem;
the collection vessel includes a central column defining an opening; and
the second end of the actuating stem is received in the opening of the central column.

17. The device of claim 16, further comprising a cap coupled to the device housing, wherein:
the cap includes a central channel for receiving the actuating stem;
the shape of the cross-section of the middle portion of the actuating stem is one of an oval, a triangle and a square; and
the actuating stem comprises a coupler at the first end, the coupler adapted to connect the actuating stem to a motor for motorized operation or to a crank for manual operation.

18. A method of disaggregating tissue using a trituration device, the device including a device housing, a rotor block comprising a solid cylinder including a central channel and at least one recess that defines a rotor blade, a trituration grater including multiple angled cutting holes, a collection vessel to collect disaggregated tissue, and an actuating stem, the method comprising:
positioning a tissue specimen within the at least one recess of the rotor block;
inserting the rotor block into the device housing to contact the trituration grater, while the tissue specimen remains in the at least one recess of the rotor block;
inserting the actuating stem through the central channel of the rotor block; and
rotating the actuating stem using a motor or crank to rotate the rotor block and press the tissue specimen against the trituration grater to receive disaggregated tissue in the collection vessel.

19. The method of claim 18, wherein the device includes a cap having at least one side channel and at least one plug received in the at least one side channel, the method further comprising:
coupling the cap to an end of the device housing opposite the collection vessel;
removing the at least one plug from the at least one side channel;
injecting a buffer material through the at least one side channel to facilitate trituration of the tissue specimen; and
replacing the at least one plug after injection to maintain sterility of the device during operation.

20. The method of claim 18, further comprising:
placing multiple beads in the collection vessel and shaking the collection vessel after receiving the disaggregated tissue to facilitate breaking apart of the disaggregated tissue; and/or
placing one or more tissue disaggregation agents in the collection vessel to provide tissue disaggregation for tissue specimens received in the collection vessel.
